(19) United States
(12) Patent Application Publication
(10) Pub. No.: US 2005/0241013 A1
(43) Pub. Date: Oct. 27, 2005

(54) Unique use of sHLA-G obtained in soluble form from JEG-3 cell line, by purification by PCR, HPLC, or any other techniques, as well as in other forms, as an implantation promoting agent when added to embryo culture and/or to the media in which embryos are transferred to the uterus following in vitro fertilization

(76) Inventor: Geoffrey Sher, Las Vegas, NV (US)

Correspondence Address: Geoffrey Sher;Attn: D. Kemp, c/o Inova Management Company, 3121 S. Maryland Parkway, Suite 206, Las Vegas, NV, 89109, US

(21) Appl. No.: 10/829,081

(22) Filed: Apr. 22, 2004

(51) Int. Cl. C12N 5/08

(52) U.S. Cl. 800/21; 435/366

(57) ABSTRACT

Survival of the fetal allograft is in large part dependent upon the establishment of a “harmonious interaction” between the trophoblast and decidual lymphocytes. It has been suggested that upon arrival at the site of implantation, a novel gene of non-classical human leukocyte antigen (HLA) class I antigen, HLA-G, produced predominantly by the extravillous cytotrophoblast (which represents the only fetal cells that are in direct contact with maternal decidual cells) immediately signals decidual lymphocytes. In response, these lymphocytes release growth factors (cytokines), initiating a “cross-talk” with the embryo, referred to as the cytokine network. It is this “dialogue” that is believed to establish and promote implantation. The detection of sHLA-G in embryo culture media of grouped and single embryos that are most likely to implant suggests that sHLA-G may have a role in optimizing implantation potential in IVF procedures. sHLA-G has recently been isolated from the culture media surrounding pooled developing embryos and blastocysts. It has been observed that the absence of sHLA-G in the supernatant surrounding groups of embryos in culture is associated with significantly reduced IVF implantation and pregnancy rates. I propose that addition of sHLA-G to the medium in which embryos are cultured and/or delivered into the uterine environment though embryo transfer, will enhance implantation and pregnancy potential of those embryos.

This patent application is for the unique use of sHLA-G obtained in soluble form from JEG-3 cell line, by purification by PCR, HPLC, or any other techniques, as well as in other forms, as an implantation promoting agent when added to embryo culture and/or to the media in which embryos are transferred to the uterus following in vitro fertilization.

### CROSS-REFERENCE TO RELATED APPLICATIONS

Not Applicable

### STATEMENT REGARDING FEDERALLY SPONSORED RESEARCH OR DEVELOPMENT

Not Applicable

REFERENCE TO SEQUENCE LISTING, A TABLE, OR A COMPUTER PROGRAM LISTING COMPACT DISK APENDIX

Not Applicable

### BACKGROUND OF THE INVENTION

HLA-G was cloned in 1987. This protein is quite different from classical HLA class I antigens (A, B, and C) in that it is almost monomorphic and the site of expression is extremely limited. Soluble human leukocyte antigen (sHLA) class I molecules have been recognized since 1970, but only recently have they have become the subject of intense research because of their presumed importance in the immune response and in the modulation of the maternal-fetal immune relationship during pregnancy. sHLA-G was first described as a major histocompatability complex (MHC) class I b gene exhibiting a very restricted tissue distribution limited to extravillous cytotrophoblast cells in the placenta, as well as in maternal spiral arteries, endothelial cells of fetal vessels in the chorionic villi, amnion cells, thymus, and on interferon-γ-stimulated blood monocytes. So far, all of the data demonstrate that the in vivo sHLA-G protein expression is restricted to the maternal-fetal interface and thymus. Moreover, the sHLA-G molecule is strongly expressed during the first trimester of gestation and then decreases through the remainder of pregnancy, suggesting its role in implantation and its protective function during pregnancy.

sHLA-G molecules have been shown to display six alternative splicing products, four of which encode different truncated extracellular domains, as two products, soluble sHLA-G1 and -G2 which lacks exons 5 and 6, but contain intron 4. The resulting isoforms are likely to be expressed in soluble form, since they lack the transmembrane and intracellular domains. One of these transcripts encodes the full-length sHLA-G1 soluble form and yields a 37-kDa soluble protein that lead to a purified form of sHLA-G and monoclonal antibodies, respectively.

ABSTRACT Submitted to ESHRE Congress—June 2004.

Preliminary study (2003) sHLA-G in embryo culture media: A sensitive Vindicator of “embryo competency” and IVF outcome.

G. Sher^{1,2}, L. Keskintepe¹
¹Sher Institutes for Reproductive Medicine (SIRM), Las Vegas, Nev., ²Department of Obstetrics and Gynecology, University of Nevada School of Medicine, Reno Nev.

Soluble HLA-G (sHLA-G) has been isolated from the culture media surrounding embryos and blastocysts. The absence of sHLA-G from human embryo culture media is associated with reduced embryo development and pregnancy rates. We sought to determine whether expression of sHLA-G in media surrounding individually cultured embryos could be used as a “marker”, that could predict subsequent IVF outcome.

The media surrounding 397 individual ICSI-derived embryos in 106 women (26-43 years, mean=36.9±5.8) were evaluated for sHLA-G expression. Ninety four women (28-44 years, mean=35.7±5) received embryos derived from their own oocytes. Seventy were less than 40 years while 24 were 39-44 years. Twelve women (38-54 years, mean 43.8±4.0) received fresh embryos derived from donated oocytes (donors aged 22-34 years, mean=24.7±3.7). Each embryo was cultured in 50 μl of P-1 medium for 46 hours. Samples of the media were immediately frozen, stored, and later thawed for an ELISA test to measure sHLA-G expression. Three hundred and two of 397 embryos derived from both patients own eggs and donated eggs were transferred (mean=2.9) to 102 women within 72 hours of egg retrieval (four cases were canceled prior to ET).

sHLA-G concentration OD<0.157±0.056 in the media was defined as “negative” expression, while a mean value of OD≧0.253±0.056 was defined as “positive”.

Results: Thirty eight of the 102 patients (37%) achieved one or more ultrasound confirmed clinical pregnancies. Group 1 (n=49) had at least one embryo transferred (mean=3.1) that tested “positive” for sHLA-G. The clinical pregnancy and implantation rate per ET were 78% ( 38/49) and 36% ( 54/152), respectively. The multiple pregnancy rate was 10% ( 4/38) when only one sHLA-G “positive” embryo was transferred and, 32% ( 12/38) when more were transferred. In Group 2 (n=53) none of the embryos transferred (mean=3.2) tested “positive” for sHLA-G. Six had clinical pregnancies 11% ( 6/53). The implantation rate per ET was 7% ( 10/145).

### APPLICATIONS

IVF in natural ovulatory cycles.
IVF performed following Controlled Ovarian Hyperstimulation with Gonadotropins, Clomiphene Citrate or with any other drugs that stimulate ovarian follicle development
During Frozen Embryo Transfer (FET) recipient cycles.
During Ovum Donation, Embryo Adoption, Gestational Surrogacy Embryo Recipient Cycles.

### REFERENCES

1. Menicucci A, Noci I, Fuzzi B, Criscuoli L, Baricordi O, Mattiuz P I: Non-classic sHLA class I in human oocyte culture medium. Hum Immunol 60:1057, 1999.
2. Fuzzi B, Rizzo R, Criscuoli L, Noci I, Melchiorri L, Scarselli B, Bencini E, Menicucci A, Baricordi O: HLA-G expression in early embryos is a fundamental prerequisite for the obtainment of pregnancy. Eur J Immunol 32:311, 2002. ³. Van Rood J J, Van Leeuwan A, Van Santen M C T: Anti-HLA-A2 inhibitor in normal human sera. Nature 226: 336, 1970.
4. Haga J A, She J X, Kao K J: Biochemical characterization of 39 kDa class I histocompatibility antigen plasma. A secretable membrane protein derived from transmembrane domain deletion. J Biol Chem 266:3695, 1991.
5. Fournel S, Aguerre-Girr M, Campan A, Salauze L, Berrebi A, Lone Y C, Lenfant F, Bouteiller P: Soluble HLA-G: Purification from eucariotic transfected cells and detection by a specific ELISA km J Reprod Immunol 42:22, 1999.
6. Fournel S. Huc X, Aguerre-Girr M, Solier Q, Legros M, Proud-Brethenou C, Moussa M, Chaouat G, Berrebi A, Bensussan A, Lenfant F, Le Bbouteiller P: Comparative reactivity of different HLA-G monoclonal antibodies to soluble HLA-G molecules. Tissue Antigens 55:510, 2000.
7. Van Lierop M, Wijnands F, Loke Y, Emmer P, Lukassen H, Braat D, Van der Meer A, Mosselman S, Josten I: Detection of HLA-G by specific sandwich ELISA using monoclonal antibodies G233 and 56B. Mol Hum Reprod 776, August 2002.
8. Loke Y W, King A: Human Implantation: Cell Biology and Immunlogy. Cambridge, Cambridge University Press, 1995
9. Hunt J S: HLA and maternal-fetal relationship. Austin, Tex. RG Landes Co, 1996.
10. Blaschitz A, Lenfant F, Mallet V, Hartmann M, Bansusan A, Geraghty D E, Le Bouteiller P, Dohr G: Endothelial cells in chorionic fetal vessels of first trimester placenta express HLA-G. Eur J Immunol 27:3380, 1997.

### BRIEF SUMMARY OF THE INVENTION

sHLA-G has recently been isolated from the culture media surrounding pooled developing embryos and blastocysts. It has been observed that the absence of sHLA-G in the supernatant surrounding groups of embryos in culture is associated with significantly reduced IVF implantation and pregnancy rates. We propose that addition of sHLA-G to the medium in which embryos are cultured and/or delivered into the uterine environment through embryo transfer, will enhance implantation and pregnancy potential of those embryos.

### BRIEF DESCRIPTION OF THE SEVERAL VIEWS OF THE DRAWING

Not Applicable

### DETAILED DESCRIPTION OF THE INVENTION

Following in vitro fertilization of oocyte(s) by intracytoplasmic sperm injection (ICSI), the embryo transfer media containing 0.200-0.250 OD sHLA-G will be used for a brief embryo culture (10 min), after which the embryos and media will be loaded into the transfer catheter and subsequently, transferred to the uterus. Initially, sHLA-G will be used in a concentration of 0.200-0.250 OD, however, serial dilution experiments will be conducted in order to identify the optimal concentration for clinical application.

1. I claim that adding sHLA-G to the embryo transfer media may improve endometrial receptivity by signaling immune cells, including Natural Killer cells, T & B lymphocytes, macrophages, and monocytes in the endometrium to facilitate immune acceptance of the embryonic semi-allograft and thus, potentially augment embryo implantation and hence, pregnancy potential subsequent to in vitro fertilization.
